(19) United States
(12) Patent Application Publication
(10) Pub. No.: US 2025/0375268 A1
(43) Pub. Date: Dec. 11, 2025

(54) FACE AND AIRWAY DEVELOPER APPLIANCE AND METHOD

(71) Applicant: Joseph YOUSEFIAN, Bellevue, WA (US)

(72) Inventor: Joseph YOUSEFIAN, Bellevue, WA (US)

(21) Appl. No.: 19/179,964

(22) Filed: Apr. 15, 2025

Related U.S. Application Data
(63) Continuation of application No. 16/729,551, filed on Dec. 30, 2019, now Pat. No. 12,274,596.
(60) Provisional application No. 62/843,089, filed on May 3, 2019.

(51) Int. Cl. A61C 7/06; A61C 7/36; A61F 5/56

(52) U.S. Cl. CPC A61C 7/06; A61C 7/36; A61F 5/56; Y10S 602/902

(57) ABSTRACT

A facemask and accessories which overcome the adverse effects and limitations of current designs by moving the maxilla and mandible in forward direction, which assists in correcting maxillary and mandibular skeletal and dentoalveolar deficiency and help expand the upper airway by advancing the mandible. This is accomplished by a design that is supported only by forehead and produces forces in front of maxilla and mandible. The extra-oral facebow design is characterized by providing two independent force adjustments, one above the forehead and the other one in front of the face by attachments to the upper and lower jaw bones and teeth. In particular, the two independent adjustments are provided by a fully adjustable forehead pad and bilateral adjustable bows on opposite sides of the facebow's extra-oral frame to provide protracting forces.

### CROSS-REFERENCES

The following applications and materials are incorporated herein by reference, in their entireties, for all purposes: U.S. Provisional Patent Application Ser. No. 62/843,089, filed May 3, 2019; and U.S. patent application Ser. No. 16/729,551, filed Dec. 30, 2019.

### INTRODUCTION

Orthodontists, dental practitioners and medical-dental researchers are constantly searching for new and improved ways to correct the problem of short and small jaws that can contribute to an unaesthetic facial appearance and the development of airway related disorders such as obstructive sleep apnea. This condition, by a narrowing of the tongue space and a retraction of the tongue back to the airway can cause the constriction of the upper airway in the retropalatal (behind the palate), retroglossal (behind the tongue) and hypoglossal (behind and below the tongue) areas. As a result, this condition not only causes unaesthetic small and retruded jaws and adversely affects the facial profile, but also causes the upper airway of the patient to become constricted and thereby resist air passage especially during the deeper stage of sleep when the upper airway muscles relax and cannot provide ideal support for the patency of the airway.

In the past, many different methods have been used in order to address retruded jaws and their effect on the airway. One method that has been utilized by orthodontic practitioners is that of dental expansion of the upper and lower dental arch, combined with dental and/or orthopedic expansion of the upper jaw in transverse direction, followed by forward protraction of the upper and lower jaws by a reverse pull facemask. In this protocol, the face mask utilizes the anchorage from different areas of the face including the combination of the patient's forehead and chin (transferring the force to the body of the mandible and to the temporomandibular joints (TMJ) which are growth sites or centers in children and adolescents still growing [henceforth, “children”]), forehead and zygomatic processes which consists of zygomatic bones and maxillary bone supporting the midface and upper airway of the patient.

The forehead is a stable structure that cannot receive an adverse effect on its growth. However, the use of zygomatic bones or chin can have deleterious effect on the growth and development of these components, especially maxillary and mandibular bones that are attached to them, which can stop or slow down the growth of the upper and lower jaws in children.

In children, force application to the chin by reverse-pull headgear causes downward and backward rotation of the mandible. Although the retrusive forces being applied by the chin cup can benefit a prognathic mandible, it may be detrimental to the TMJ as well as increase open bite tendencies.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

The appliance and method of the present disclosure is distinguished from conventional facemasks by avoiding pressure on the zygomatic, maxillary and mandibular bones and TMJs. This invention is characterized by providing extra oral forces supported by the forehead of the patient to pull maxillary and mandibular bones, dentition, and tongue forward and as result help to grow the upper and lower jaw structures also open the airway of the patient by connection to the lower jaw of the patient.

This face and airway developer design and method according to the disclosure overcomes the adverse effects and limitations of current designs by avoidance of transferring anchorage forces to the zygomatic, maxillary, mandibular and TMJ structures and helps pulling the maxillary, mandibular bones and dentition effectively forward to enlarge the retropalatal, retroglossal and hypoglossal airways.

This is accomplished using a design that positions a pad over the forehead of patient as forehead rest and attached to the frame as type 1 lever system that on one end above the forehead attached to the back of the skull for anchorage by adjustable or fixed, non-elastic or elastic straps that produces forces that transferred over the fulcrum to the other end of the frame and lever in front of the face or mouth of the patient. From this point of attachment, elastics, non-elastic straps or attractive magnetic elements can be extended to the upper and lower jaws by means of brackets, bands attached to the teeth, or via clear aligners, face bows, expanders, bone implants, soft tissue anchors secure to the maxillary and mandibular teeth or bones or tongue.

The device can include an intra-oral frame adapted to be connected to the patient's teeth and an extra-oral frame connected at one end to the intra-oral frame or other connections and connected at the other end to the frame that extend to the forehead rest or face bow attached to it connected via elastic or non-elastic means to the back of the head. The extra-oral design of the invention is characterized by providing adjustments (forehead width and horizontal/vertical dimensions) that allow the invention to be customized to each patient. In particular, the two independent adjustments are provided, one by elastics or non-elastic straps secured above the forehead extended directly or via a face bow frame around the base or back of the patient's head. The other one to the lever arm in front of the face and mouth that is connected intraorally to the teeth or bones or tongue of patient.

This is accomplished using a design that produces forces above the center of resistance of the maxilla and parallel to the Frankfort horizontal plane.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a side elevational view of a face and airway developer appliance, according to the present invention, installed onto the face of a human patient, showing the extra-oral portion of the appliance.

FIG. 2 is a front elevational view of the appliance of FIG. 1, also shown installed onto the face of a human patient.

FIG. 3 is a plan side view showing an alternative apparatus for correcting malocclusion and growing the lower jaw.

FIG. 4a is a plan side view of a portion of the face and airway developer of FIG. 1, showing the intraoral portion of one embodiment, in its environment attached inside a patient's mouth.

FIG. 4b is a bottom view of a maxillary expander with flexible loops and non-parallel expansion screws attached to the roof of the mouth by mini-implants and bonded to the top of the teeth; and having extension arms to attach to the assembly of FIG. 1 with straps.

FIG. 5 is a plan side view of a portion of the face and airway developer of FIG. 1, showing the intraoral portion of an alternate embodiment, in its environment attached inside a patient's mouth.

FIG. 6 is a plan side view of a portion of the face and airway developer of FIG. 1, showing the intraoral portion of another alternate embodiment designed to apply torque to an upper molar, in its environment attached inside a patient's mouth.

FIG. 7a is plan side view of a portion of the face and airway developer of FIG. 1, showing the intraoral portion of yet another alternate embodiment adapted to treat sleep apnea, in its environment attached inside a patient's mouth to the lower jaw and tongue.

FIG. 7b is a plan view of the appliance of FIG. 7a, with the tongue and lower jaw pulled forward to open the airway.

FIGS. 8a and 8b show the appliance of FIGS. 7a and 7b, but in an environment attached inside the mouth of a patient lacking teeth.

### DETAILED DESCRIPTION

Definition: the term “teeth” as used in this application encompasses implants, and any other structure that can deliver a force to the jaw bones, such as an implanted anchor for a mating element.

The term “child” refers to patients whose facial features are still experiencing growth and encompasses adolescents who are still growing.

A “strap” is any element capable of being placed into tension, and thereby to transfer a pulling force. A “strap” may be elastic or inelastic.

An “intra oral feature” of a patient, is the teeth, as defined above, or the jaws or tongue.

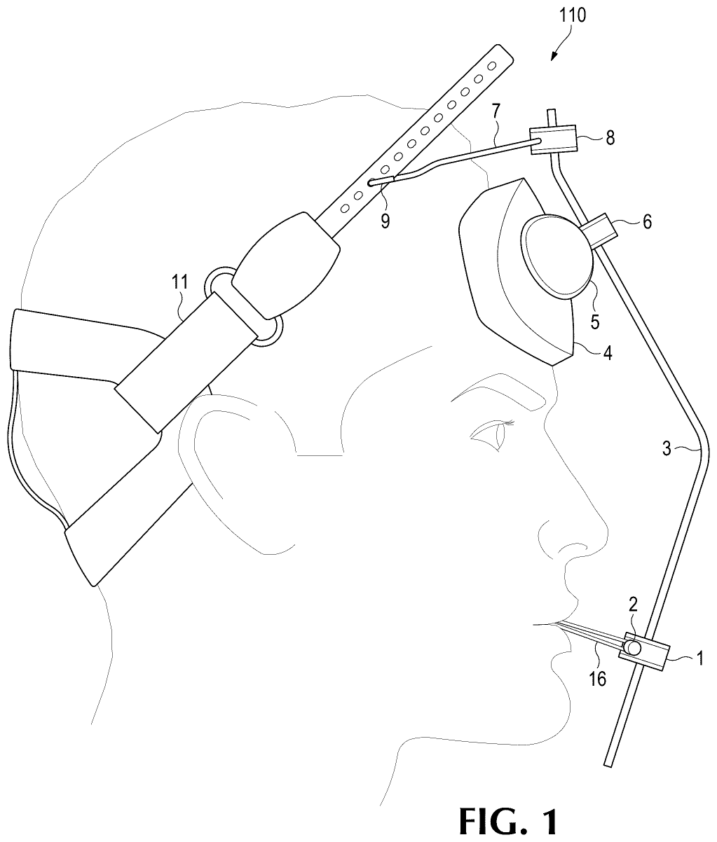

Referring to FIG. 1, a face and airway developer appliance 110, includes an extra-oral assembly that consists of a lever 3 in the form of a vertical, bow shaped metal rod, slidingly engaged to a fulcrum block 6 that is supported by a detachable fulcrum support 5. The fulcrum support 5 is supported solely by cushions 4 contacting the patient's forehead. The appliance includes a bottom block 1, slidingly supported on lever 3 in front of a patient's mouth, which in turn supports a tooth pull bar 2, for attachment via tooth straps 16 to a patient's intra-oral features.

One embodiment has a lever pull bow 7 connected to the lever 3 by a top block 8 which pulls the top of lever 3 toward the forehead, causing bar 2 to be pushed away from the jaws. Block 8 can slide on lever 3, to permit proper moment arm force delivery to the lever 3. The bow 7 which extends laterally to the either side of patient's forehead, ends with hooks 9 for connection to a set of head straps 11 that extend on either side of the face of the patient and around the back of the patient's skull. Strap set 11 is adjusted to pull on hooks 109, and thereby pull back on lever 3.

The adjustment blocks 1, 6, 8 permit customization to each patient, as they can each be slid up or down the lever 3 to adjust the force delivery from straps 11.

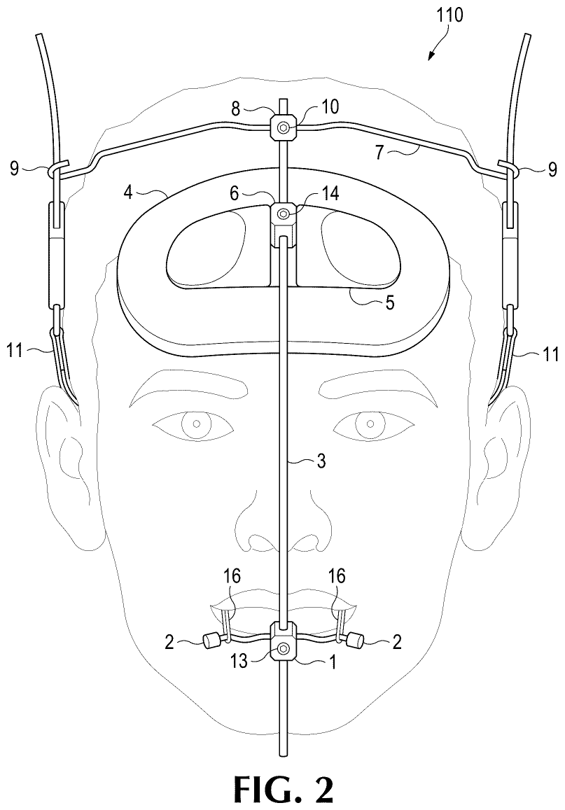

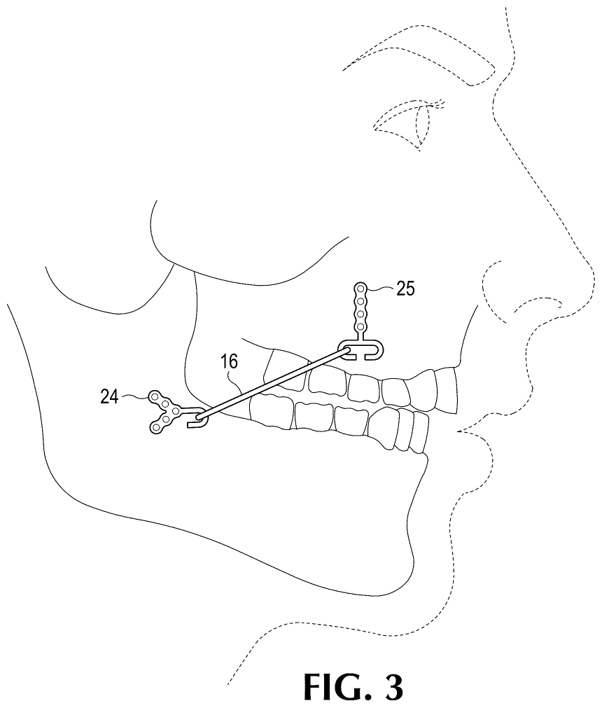

A set screw 13, 10, 14 (FIG. 2) in block 1, 6, 8 respectively, are used to fix the vertical location of the bar 2 and bow 7 and fulcrum support 5 on the lever 3.

The vertical position of the block 1 and horizontal bar 2 on the direction of the force delivery to the upper or lower jaws based on the clinical requirement for normalization of the facial structures for proper growth and airway structures for normalization of the patient's airway by advancing the mandible

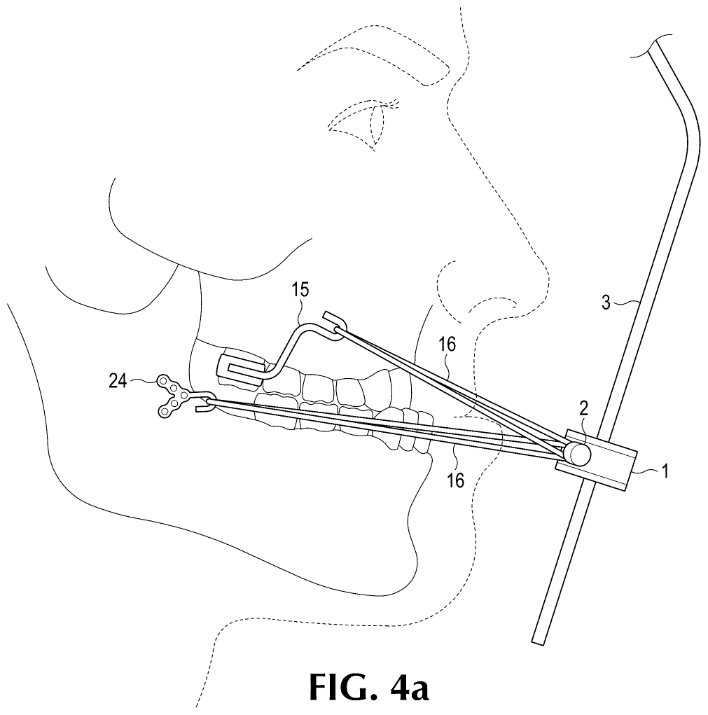

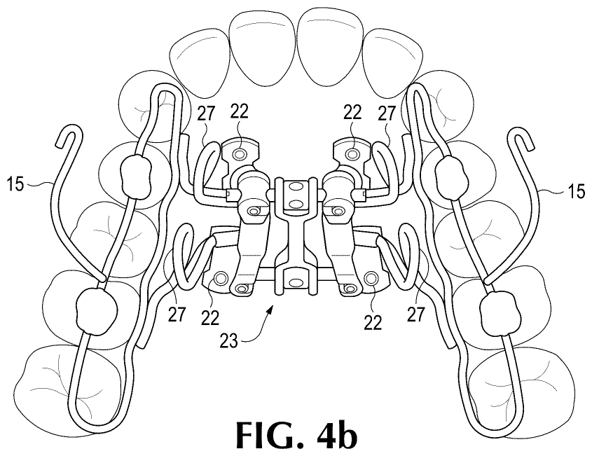

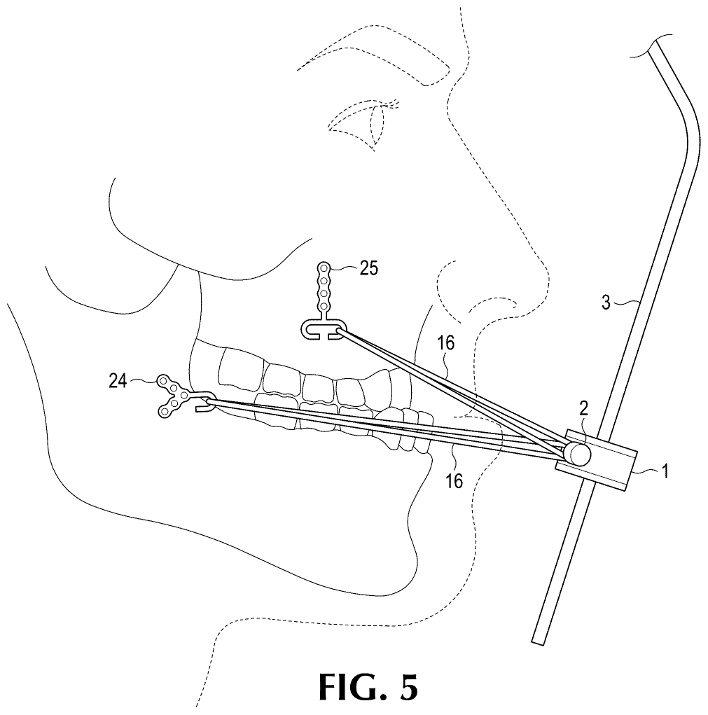

Referring to FIGS. 4a and 4b, device 110 further includes an intra-oral portion, including an intra-oral portion of straps 16, which are attached to anchors 15 of a maxillary anchoring assembly 23. In child patients, pulling on anchors 15 loosens the maxillary connection structures to the base of skull and causes a forward expansion of the upper jaw, which is highly desirable in patients with maxillary sagittal deficiency. By adjusting the force applied, the vertical growth of the maxilla can be encouraged, which is desirable in patients with vertical maxillary deficiency. Referring to FIG. 4b, maxillary anchoring assembly 23 includes screws 22, which are driven into the patient's palate. Screws 22 may be selectively removed on one side to provide a greater pulling force on the teeth on one side of the maxilla, than another. Side screws attached to the bone, make the maxillary bone on that side be pulled forward to take care of dental versus skeletal asymmetries. For example, if the patient's maxilla lacks bilateral symmetry, the left or the right side can be tightened to bone on the shorter side rather than on the other side, which is normal, to pull the short side more than the other. Also, if it is desired to pull more on the front of the maxilla, the frontmost screws 22 may be installed, rather than the rear screws 22. If the converse is desired the rear screws 22 may be installed, rather than the front screws. As shown in FIG. 4a, a set of straps 16 may be attached to mandible anchor 24. FIG. 5 shows a similar configuration, except for that assembly 23 is not shown, and a different form of maxillary anchor 25 is pulled by straps 16.

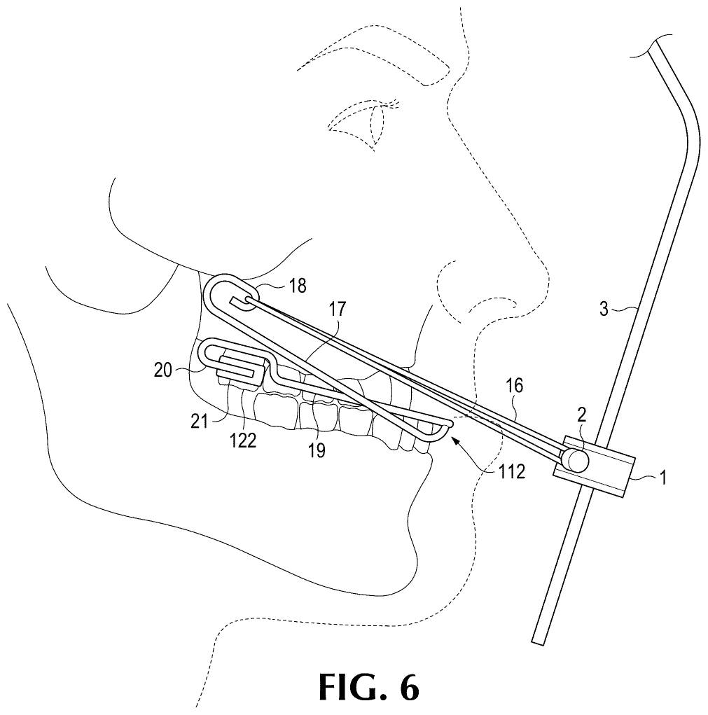

As shown in FIG. 6, a set of straps 16 pull on a structure 112, having an extra-oral and intra-oral portion, and designed to apply a vertical torque on a pair of upper molars 21. Straps connect to hooks 18 supported by extra oral wing 17 (an equal bow 17 present on the other side of the face), which in turn is connected to intra oral bow 19. Bow 19 bends at its ends 20, and each end is fit into a tube 21 that is connected to a molar 122. The forward pull on straps 16, causes a downward pull at the front of bow 19, which in turn provides a clockwise rotational force on molars 122 (in the plane of FIG. 6), accompanying the generally forward force, created by the pull of straps 16 (by way of bar 2 and bow 3). In a closely related embodiment extra oral wings 17 are placed in an intra oral position between the cheeks and the jaws.

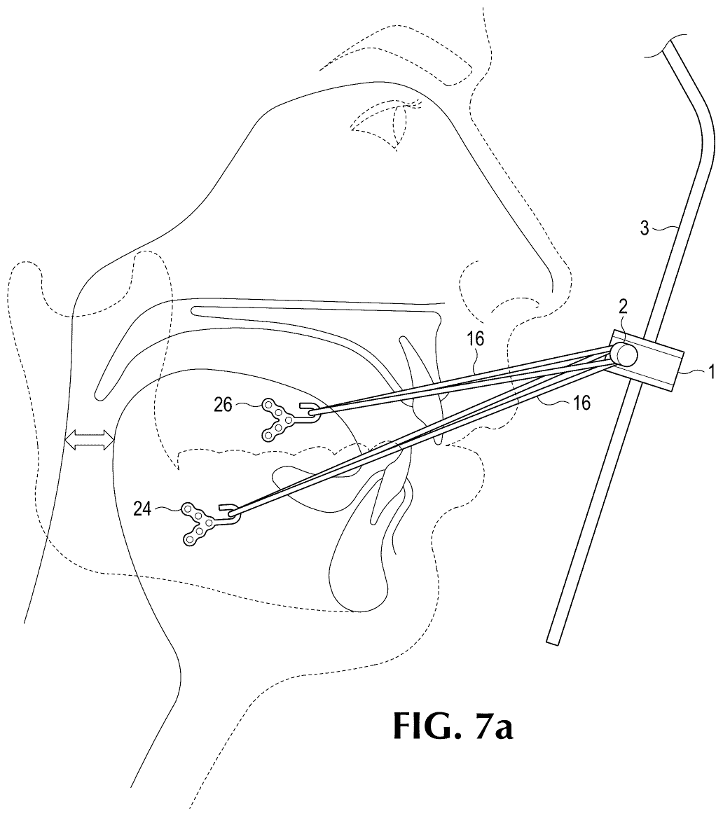

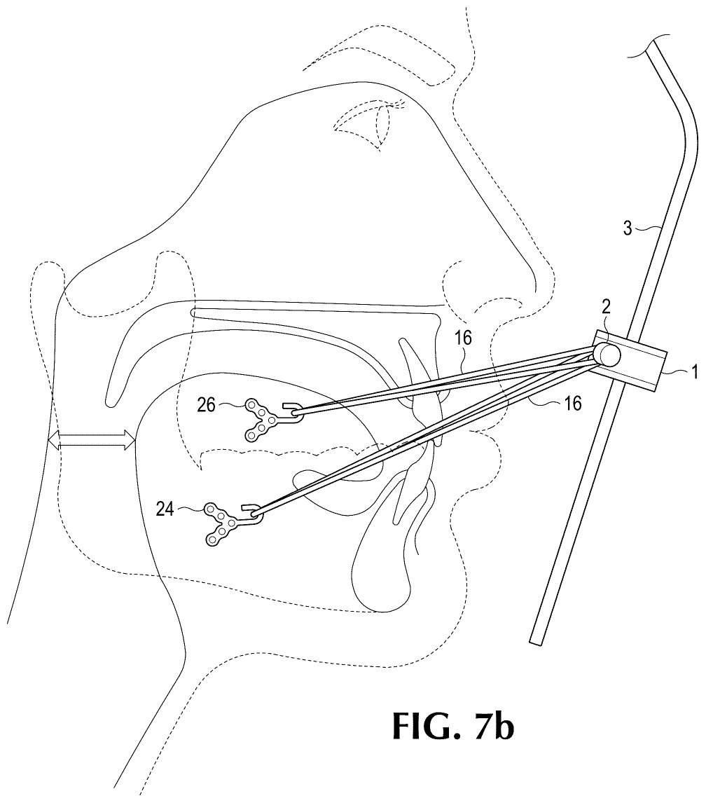

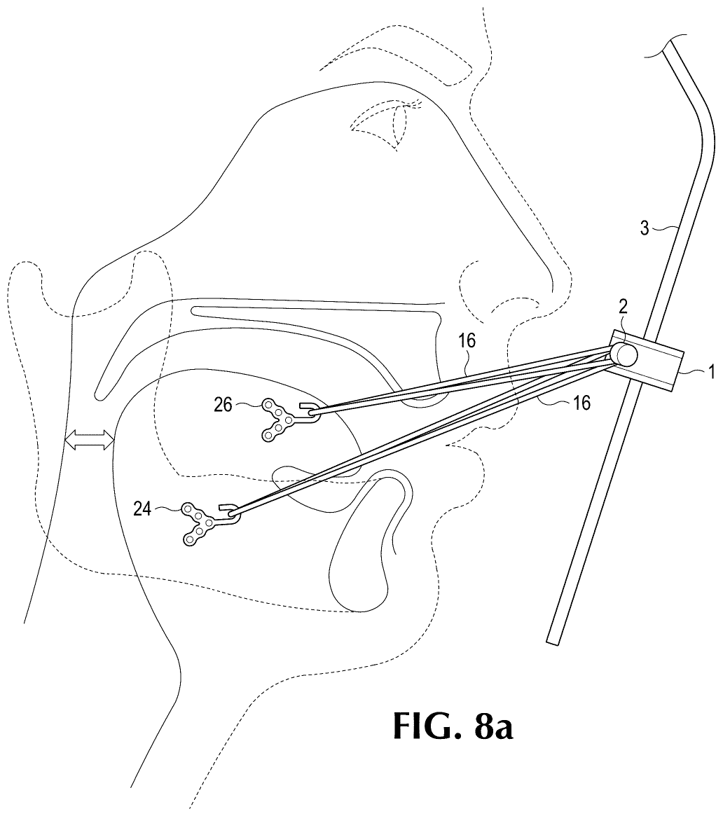

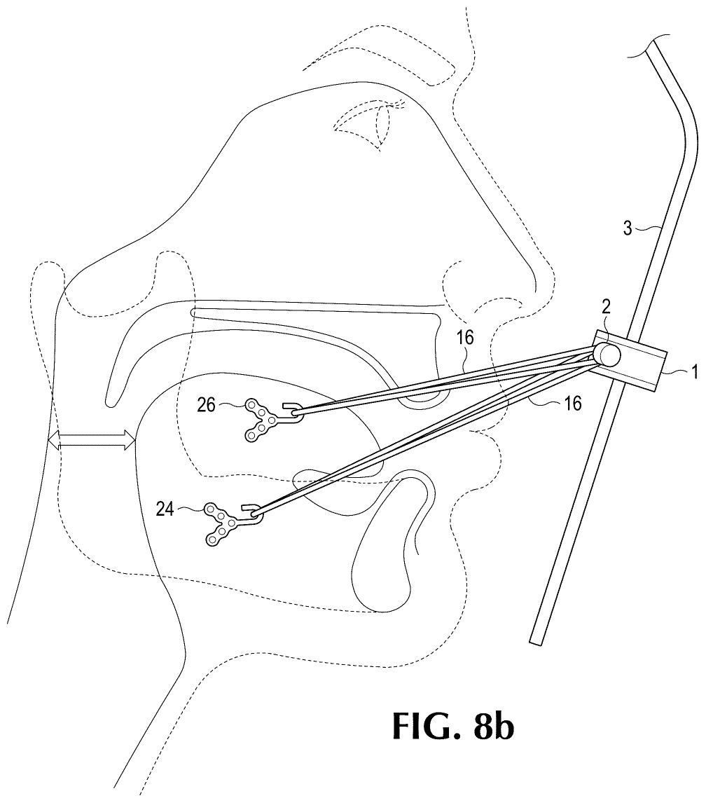

FIGS. 7a and 7b show a system in which the straps 16 are used to pull the tongue (by way of anchor 26) and the lower jaw (by way of anchor 24) forward, for the treatment of small jaws in children or sleep apnea in children or adults. FIGS. 8a and 8b show the same system but attached in a patient without teeth.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A method for treating a patient's retruded jaw, comprising:
placing a fulcrum of an appliance on a forehead of the patient, wherein the appliance comprises:
an extra-oral portion comprising a lever including the fulcrum and a rigid lever arm slidingly attached to the fulcrum, wherein the rigid lever arm has a first end disposed above the fulcrum and a second end disposed below the fulcrum, wherein the extra-oral portion further includes a headstrap set attached to the rigid lever arm at a first location disposed above the fulcrum and adjacent to the first end; and
an intra-oral strap assembly connected to the rigid lever arm at a second location disposed below the fulcrum and adjacent to the second end of the rigid lever arm;
placing the headstrap set on a head of the patient;
attaching the intra-oral strap assembly to a set of teeth of the patient; and
forcing the second end and the second location of the rigid lever arm away from the head by adjusting the headstrap set to pull on the first location of the rigid lever arm disposed above the fulcrum, such that the intra-oral strap assembly is placed into tension to pull on the set of teeth of the patient.

2. The method of claim 1, further comprising placing a pad between the fulcrum and the forehead of the patient.

3. The method of claim 1, further comprising adjusting a position of the first location on the rigid lever arm, such that the headstrap set is attached to the rigid lever arm at an adjusted first location disposed above the fulcrum.

4. The method of claim 3, further comprising securing the headstrap set to the rigid lever arm at the adjusted first location.

5. The method of claim 1, further comprising adjusting a position of the second location on the rigid lever arm, such that the intra-oral strap assembly is attached to the rigid lever arm at an adjusted second location disposed below the fulcrum.

6. The method of claim 5, further comprising securing the intra-oral strap assembly to the rigid lever arm at the adjusted second location.

7. The method of claim 1, wherein the set of teeth includes one or more upper teeth of the patient.

8. The method of claim 1, wherein the set of teeth includes one or more lower teeth of the patient.

9. An oral treatment appliance, comprising:
an extra-oral portion, including;
a fulcrum piece configured to be placed on a forehead of a patient;
a lever slidingly attached to the fulcrum piece and having a first end disposed above the fulcrum piece and a second end disposed below the fulcrum piece, wherein the second end is opposed to the first end; and
a headstrap set coupled to the lever at a first location disposed above the fulcrum piece and adjacent to the first end, wherein the headstrap set is configured to be placed on a head of the patient; and
an intra-oral strap assembly configured to be attached to an intra-oral feature of the patient, wherein the intra-oral strap assembly is connected to the lever at a second location disposed below the fulcrum piece and adjacent to the second end, wherein the headstrap set is configured to pull on the first location of the lever disposed above the fulcrum piece, such that the second end and the second location of the lever are forced away from the head of the patient and the intra-oral strap assembly is placed into tension to pull on the intra-oral feature of the patient.

10. The oral treatment appliance of claim 9, wherein the headstrap set includes:
a headstrap block coupled to the lever at the first location disposed above the fulcrum piece;
a pair of hooks coupled to the headstrap block;
a pair of arms each defining a set of apertures, wherein each aperture of the set of apertures is configured to receive a respective one of the hooks to couple the respective one of the hooks to the arm; and
a headstrap coupled to the pair of arms and configured to extend around a back of the head of the patient between the pair of arms, wherein the headstrap set is configured to be adjusted by connecting the pair of hooks to different apertures of the set of apertures of each arm.

11. The oral treatment appliance of claim 9, further including a fulcrum support configured to be disposed between the fulcrum piece and the forehead to support the fulcrum piece on the forehead.

12. The oral treatment appliance of claim 9, wherein the intra-oral strap assembly is connected to the lever by a connection block, wherein the connection block is configured to slide on the lever, and wherein the connection block is configured to be locked in a selected position on the lever using a fastener.

13. The oral treatment appliance of claim 9, wherein the intra-oral strap assembly includes one or more straps and attachment elements configured to attach the one or more straps to the intra-oral feature.

14. The oral treatment appliance of claim 9, wherein the intra-oral feature is one or more upper or lower teeth of the patient.

15. The oral treatment appliance of claim 9, wherein the intra-oral feature is an upper or lower jaw of the patient.

16. The oral treatment appliance of claim 9, wherein the intra-oral strap assembly includes:
a connection block coupled to the lever below the fulcrum piece at the second location;
an intra-oral bow having a pair of rearward ends each configured to be coupled to the intra-oral feature of the patient;
a pair of wings each including a lower-front end and an upper-rear end, wherein the lower-front end of each wing is coupled to the intra-oral bow, and wherein each wing terminates in a hook at the upper-rear end of the wing; and
a pair of straps connecting the hook of each wing to the connection block, such that the pair of straps are configured to pull forward and downward on the hook when the intra-oral strap assembly is placed into tension.

17. A method of re-forming a maxilla of a patient, comprising:
attaching an intra-oral bow of an oral treatment appliance to a pair of molars of the patient, such that the intra-oral bow is configured to apply a vertical torque on the pair of molars, wherein the oral treatment appliance further includes:
a pair of wings each including a lower-front and an upper-rear end, wherein the lower-front end of each wing is coupled to the intra-oral bow, and wherein each wing terminates in a hook at the upper-rear end of the wing; and
attaching the hook of each wing to a respective strap configured to pull forward and downward on the hook.

18. The method of claim 17, wherein the pair of wings are configured to be disposed external to a mouth of the patient.

19. The method of claim 17, wherein each wing extends rearward and upward from the lower-front end to the upper-rear end of the wing.

20. The method of claim 17, further comprising:
placing a fulcrum of the oral treatment appliance on a forehead of the patient, wherein the oral treatment appliance further comprises:
an extra-oral portion comprising a lever including the fulcrum and a rigid lever arm slidingly attached to the fulcrum, wherein the rigid lever arm has a first end disposed above the fulcrum and a second end disposed below the fulcrum, wherein the extra-oral portion further includes a headstrap set attached to the rigid lever arm at a first location disposed above the fulcrum and adjacent to the first end; and
a lower sliding block secured to the rigid lever arm at a second location disposed below the fulcrum and adjacent to the second end;
placing the headstrap set on a head of the patient;
attaching each respective strap of the oral treatment appliance to the lower sliding block; and
forcing the second end and the second location of the rigid lever arm away from the head of the patient by adjusting the headstrap set to pull on the first location of the rigid lever arm disposed above the fulcrum, such that each respective strap of the oral treatment appliance is placed into tension to pull forward and downward on the hook of each wing of the oral treatment appliance.
